(19) United States
(12) Patent Application Publication
(10) Pub. No.: US 2006/0280709 A1
(43) Pub. Date: Dec. 14, 2006

(54) Emollient mixture and use thereof as a mineral oil substitute

(76) Inventors: Achim Ansmann, Erkrath (DE); Bettina Jackwerth, Langenfeld (DE); Gary Dee, Blue Bell, PA (US); Stefan Bruening, Philadelphia, PA (US)

Correspondence Address: COGNIS CORPORATION;PATENT DEPARTMENT, 300 BROOKSIDE AVENUE, AMBLER, PA, 19002, US

(21) Appl. No.: 10/549,953

(22) PCT Filed: Mar. 11, 2004
(86) PCT No.: PCT/EP04/02495

§ 371 Date: Aug. 3, 2006

(30) Foreign Application Priority Data
Mar. 20, 2003 (DE) 103-12-352.0

(51) Int. Cl. A61K 8/72; A61K 8/81; A61K 8/37

(52) U.S. Cl. 424/70.11; 424/401

(57) ABSTRACT

The invention relates to an emollient mixture which is particularly suitable as a mineral oil substitute in cosmetic compositions. The emollient mixture comprises at least one ester selected from the esters of a C₈₋₁₈ fatty acid with a C₃₋₁₂ alcohol and the esters of adipic acid with a C₃₋₁₂ alcohol in combination with a poly-α-olefin, the percentage content of the ester being between 10 and 90% by weight, based on the total quantity of ester and poly-α-olefin.

### FIELD OF THE INVENTION

This invention relates to an emollient mixture which is particularly suitable as a sensory substitute for mineral oil in cosmetic preparations.

### PRIOR ART

Mineral oils of various origins are used as a basic ingredient in numerous cosmetic preparations, such as body lotions, creams and stick preparations. However, mineral oil has the disadvantage that it is not a chemically uniform material which can be exactly described and forms an oily, impermeable film on the skin. The effect of this is that moisture is trapped beneath the film and prevents the skin from breathing normally. Attempts have already been made to develop alternatives to mineral oil. Unfortunately, these alternatives have been unable to provide the same sensory properties. There is therefore a need for a mineral oil substitute in cosmetic preparations which would be comparable with mineral oil in its sensory properties but would not have any of its negative properties.

Accordingly, the problem addressed by the present invention was to find such a substitute.

### DESCRIPTION OF THE INVENTION

The problem stated above has been solved by the emollient mixture according to claim 1. Preferred embodiments can be found in the subsidiary claims. The present invention also relates to the use of the emollient mixture claimed in claim 15 and to the cosmetic preparation claimed in claim 16.

The emollient mixture according to the invention is characterized in that, besides a poly-α-olefin, it contains at least one ester selected from esters of a C₈₋₁₈ fatty acid with a C₃₋₁₂ alcohol and the esters of adipic acid with a C₃₋₁₂ alcohol. The percentage contribution of the ester to the total quantity of ester and poly-α-olefin is between 10 and 90% by weight. Accordingly, the percentage content of the poly-α-olefin is between 90 and 10% by weight.

Particularly suitable fatty acid esters are those which contain 12 to 18 carbon atoms in the fatty acid component. The alcohol component of the fatty acid ester preferably contains 3 to 8 carbon atoms. The same applies to the alcohol component of the adipic acid ester. The alcohols are preferably monoalcohols and may be branched or unbranched. The compounds may be used either individually or in combination with one another.

Particularly suitable esters are selected from di-n-butyl adipate (for example Cetiol® B, a product of Cognis Deutschland GmbH & Co. KG), ethyl hexyl cocoate (for example Cetiol® OC, a product of Cognis Deutschland GmbH & Co. KG), ethyl hexyl palmitate (for example Cegesoft® C 24, a product of Cognis Deutschland GmbH & Co. KG), ethyl hexyl stearate (for example Cetiol® 868, a product of Cognis Deutschland GmbH & Co. KG), isopropyl myristate (obtainable from Cognis Deutschland GmbH & Co. KG) and isopropyl palmitate (obtainable from Cognis Deutschland GmbH & Co. KG).

The compounds mentioned are basically known emollients in the cosmetics field. When combined with the similarly known poly-α-olefins, they form emollient mixtures which are eminently suitable as a mineral oil substitute in cosmetics. So far as their sensory properties are concerned, the emollient mixtures according to the invention are comparable with mineral oils, but are superior in their skin-care effect. The so-called negative occlusive effect observed with mineral oils does not occur with the emollient mixtures according to the invention.

Poly-α-olefins (PAOs) are obtained by oligomerization of α-olefins which in turn are obtained by oligomerization of ethylene. The degree of oligomerization can be controlled so that poly-α-olefins differing in their molecular weights and hence in their viscosities are obtained. The double bonds still present in the poly-α-olefins after oligomerization can be hydrogenated in a subsequent step. Hydrogenated poly-α-olefins are preferably used for the purposes of the invention. The oligomers and especially the dimers of 1-decane or 1-dodecene, more especially the hydrogenated products, are particularly suitable for use in the emollient mixture according to the invention.

The choice of the poly-α-olefin depends inter alia on the intended use of the emollient mixture according to the invention. One criterion is, for example, the required viscosity of the end product. For example, poly-α-olefins with a kinematic viscosity at 100° C. of 1 to 100 cSt, preferably 1 to 40 cSt and more particularly 1.5 to 10 cSt are suitable for the emollient mixture according to the invention. PAOs such as these are obtainable, for example, from the Chevron Phillips Chemical Company LP under the name of “Synfluid®”. “Synfluid® PAO 2 cSt” is mentioned as an example of a particularly suitable representative. This product is a hydrogenated didecene with a viscosity at 100° C. of about 2 cSt.

The quantity of PAOs used in the emollient mixture according to the invention and particularly the ratio of PAO to the ester used is dependent inter alia on the type of components selected and on the intended use of the emollient mixture according to the invention. Suitable percentage contents of ester, based on the total quantity of ester and PAO, are for example 20 to 80% by weight and preferably 25 to 75% by weight. Percentage ester contents of 40 to 75% by weight are particularly suitable. Accordingly, the percentage content of PAOs, based on the total quantity of ester and PAO, is 80 to 20% by weight and preferably 75 to 25% by weight. Percentage PAO contents of 25 to 60% by weight are particularly suitable.

In a preferred variant of the invention, the emollient mixture contains no other components apart from ester and poly-α-olefins. Alternatively, however, at least one other emollient besides ester and poly-α-olefin may be used in the emollient mixture. However, the percentage content of this other emollient in the emollient mixture according to the invention does not exceed 50% by weight and is preferably no more than 30% by weight. In a particularly preferred embodiment, the mixture according to the invention contains only emollients and no other compounds.

Basically, any emollient hitherto used in cosmetic preparations may be used as the additional emollient. The addition of Guerbet alcohols or fatty acid glycerides containing 6 to 24 carbon atoms and more particularly 8 to 18 carbon atoms in the fatty acid part has proved to be particularly suitable.

The Guerbet alcohols which can be obtained by dimerization of unsaturated linear fatty alcohols and which contain an alkyl group preferably containing 2 to 18 carbon atoms in the α-position to the terminal CH₂OH group are basically known compounds. 2-Hexyl decanol, 2-butyl octanol and 2-octyl dodecanol, for example, are suitable. Preferred examples are 2-octyl dodecanol and 2-hexyl decanol which are obtainable from Cognis Deutschland GmbH & Co. KG under the names of Eutanol® G and Eutanol® G16, respectively.

Among the fatty acid glycerides, the C₈₋₁₈ fatty acid di- and triglycerides are preferred. The synthetic glycerides are normally mixtures which, besides di- and triglycerides, may also contain relatively small amounts of monoglycerides. One example of a suitable mixture of predominantly di- and triglycerides of C₈₋₁₈ fatty acids is Myritol® 331 which is obtainable from Cognis Deutschland GmbH & Co. KG. An example of a triglyceride in the chain length range of 12 to 18 carbon atoms is Cegesoft® PS 6 of Cognis Deutschland GmbH & Co. KG.

The percentage content of the additional emollient depends upon type and intended use. Suitable quantities are, for example, 0.1 to 50% by weight, more particularly 5 to 40% by weight and preferably 10 to 30% by weight of the additional emollient, based on the total quantity of ester and poly-α-olefin.

The invention is illustrated by the following Examples. The figures for the Examples set out in Table 1 below represent percentages by weight.

TABLE 1

| Constituents/ | | | | | | | | |
|---|---|---|---|---|---|---|---|---|
| Example | | | | | | | | |
| No. | 1 | 2 | 3 | 4 | 5 | 6 | 7 | C1 |
| Cetiol ® | 50.0 | 75.0 | 40.0 | 40.0 | 40.0 | 40.0 | 40.0 | — |
| OC¹⁾ | | | | | | | | |
| Isopropyl | — | — | — | 20.0 | — | — | — | — |
| palmitate²⁾ | | | | | | | | |
| Synfluid ® | 50.0 | 25.0 | 60.0 | 40.0 | 35.0 | 40.0 | 30.0 | — |
| PAO 2 cSt³⁾ | | | | | | | | |
| Myritol ® | — | — | — | — | — | — | 10.0 | — |
| 331⁴⁾ | | | | | | | | |
| Cegesoft ® | — | — | — | — | 25.0 | — | — | — |
| PS 6⁵⁾ | | | | | | | | |
| Eutanol ® | — | — | — | — | — | 20.0 | — | — |
| G⁶⁾ | | | | | | | | |
| Eutanol ® | — | — | — | — | — | — | 20.0 | — |
| G16⁷⁾ | | | | | | | | |
| Mineral oil⁸⁾ | — | — | — | — | — | — | — | 100 |

¹⁾Ethylhexyl Cocoate (Cognis Deutschland GmbH & Co. KG)
²⁾Cognis Deutschland GmbH & Co. KG
³⁾Hydrogenated Didecene (Chevron Phillips Chemical Co. LP)
⁴⁾Cocoglycerides (Cognis Deutschland GmbH & Co. KG)
⁵⁾C16-C18 Triglycerides (Cognis Deutschland GmbH & Co. KG)
⁶⁾Octyldodecanol (Cognis Deutschland GmbH & Co. KG)
⁷⁾Hexyldecanol (Cognis Deutschland GmbH & Co. KG)
⁸⁾Carnation ® While Mineral Oil (INCI: Mineral Oil, CAS 8042-47-5; WITCO)

Sensory Evaluation

The emollient mixtures prepared in Examples 2 and 4 according to the invention were compared with the mineral oil of Comparison Example C1 in sensory tests involving five trained volunteers. To this end, the mixtures were applied to the forearm of the volunteers and the following criteria were evaluated on a scale of −2 to +2: spreading (−2: difficult to +2: easy), adsorption (−2: difficult to +2: easy), tackiness (−2: high to +2: slight), oiliness (−2: high to +2: slight), waxiness (−2: high to +2: slight), velvety smoothness (−2: slight to +2: high), softness (−2: slight to +2: high) and dryness (−2: slight to +2: high). The results are set out in the following Table as averages of the individual evaluations.

TABLE 2

| | Spreading | Absorption | Tackiness | Oiliness | Waxiness | Smoothness | Softness | Dryness |
|---|---|---|---|---|---|---|---|---|
| Emollient mixture of Ex. 2 | 0 | 0 | 0.5 | 0 | 0 | 0 | 0 | 0 |
| Emollient mixture of Ex. 4 | 0 | 0.5 | 0 | 0 | 0 | 0 | 0 | 0 |
| Mineral oil of Comp. Ex. C1 | 0 | 0 | 0 | 0 | 0 | 0 | 0 | 0 |

1-16. (canceled)

17. An emollient mixture which comprises: at least one ester selected from the group consisting of esters of a C₈₋₁₈ fatty acid with a C₃₋₁₂ alcohol and the esters of adipic acid with a C₃₋₁₂ alcohol; and a poly-α-olefin wherein the content of the ester is between 10% and 90% by weight, based on the total quantity of ester and poly-α-olefin.

18. The emollient mixture as claimed in claim 17, wherein, the ester comprises a fatty acid ester containing 12 to 18 carbon atoms in a fatty acid residue.

19. The emollient mixture as claimed in claim 17, wherein, the ester comprises an ester containing 3 to 8 carbon atoms in an alcohol residue.

20. The emollient mixture as claimed in claim 19, wherein, the ester comprises at least one member selected from the group consisting of ethylhexyl palmitate, ethylhexyl stearate, isopropyl myristate and isopropyl palmitate.

21. An emollient mixture as claimed in claim 17, wherein, the poly-α-olefin comprises a hydrogenated poly-α-olefin.

22. The emollient mixture as claimed in claim 17, wherein, the poly-α-olefin comprises an oligomer.

23. The emollient mixture as claimed in claim 17, wherein the poly-α-olefin has a kinetic viscosity at 100° C. of 1 to 100 cSt.

24. The emollient mixture as claimed in claim 17, wherein, a content of the ester is 20 to 80% by weight based on the total quantity of ester and poly-α-olefin.

25. The emollient mixture as claimed in claim 17 which consists of an ester and a poly-α-olefin.

26. The emollient mixture as claimed in claim 17, wherein, the emollient contains up to 50% by weight based on a total quantity of ester and poly-α-olefin of an additional emollient.

27. The emollient mixture as claimed in claim 26, wherein, the additional emollient comprises at least one member selected from the group consisting of Guerbet alcohols and C₆₋₂₄ fatty acid glycerides.

28. The emollient mixture as claimed in claim 27, wherein, the additional emollient comprises at least one Guerbet alcohol selected from the group consisting of 2-hexyldecanol, 2-butyloctanol and 2-octyldodecanol.

29. The emollient mixture as claimed in claim 27, wherein, the fatty acid glyceride comprises at least one member selected from the group consisting of C₈₋₁₈ fatty acid diglycerides and C₈₋₁₈ fatty acid triglycerides

30. The emollient mixture as claimed in claim 26, wherein, the additional emollient is present in a quantity of 0.1 to 50% by weight, based on the total quantity of ester and poly-α-olefin.

31. A cosmetic composition comprising the emollient mixture claimed in claim 17 as a mineral oil substitute.

32. The cosmetic composition which contains the emollient mixture claimed in claim 27.

33. The emollient mixture as claimed in claim 22, wherein, the poly-α-olefin comprises at least one member selected from the group consisting of dimer of 1-decene and dimer of 1-dodecene.

34. The emollient mixture of claim 23, wherein the poly-α-olefin has a kinematic viscosity at 100° C. of from 15 to 40 cSt.

35. The emollient mixture to claim 24, wherein, the content of ester is from 25% to 75% by weight.

36. The emollient mixture of claim 30 which contains from 5% to 40% by weight of the additional emollient.

37. The emollient of claim 36 which contains from 10% to 30% by weight of the additional emollient.
